(19) United States
(12) Patent Application Publication
(10) Pub. No.: US 2009/0254082 A1
(43) Pub. Date: Oct. 8, 2009

(54) ELECTROSURGICAL INSTRUMENT

(76) Inventors: Niels Kornerup, Frorup (DK); Patrick Busch-Madsen, Slangerup (DK)

Correspondence Address: WINSTON & STRAWN LLP;PATENT DEPARTMENT, 1700 K STREET, N.W., WASHINGTON, DC, 20006, US

(21) Appl. No.: 12/373,250

(22) PCT Filed: Jul. 10, 2007
(86) PCT No.: PCT/IB07/52735

§ 371 Date: Jan. 9, 2009

(30) Foreign Application Priority Data
Jul. 10, 2006 (DK) PA 2006 00953

(51) Int. Cl. A61B 18/14; A61B 17/42

(52) U.S. Cl. 606/41

(57) ABSTRACT

The present invention relates to an electrosurgical instrument, more particularly to an electrosurgical instrument for laparoscopic hysterectomy. The instrument includes an expandable and contractible loop positioned to controllably emerge from the distal end of the shaft, with loop defining a first electrode. The instrument further includes at least one second electrode which in a first position are positioned within the shaft, and in a second position are expanded from the shaft. The instrument according to the invention is extremely simple to operate and the instrument therefore provides an improvement over prior art monopolar and bipolar treatment methods, both in decreasing treatment time, and simplifying control of the treatment.

The present invention relates to an electrosurgical instrument, more particularly to an electrosurgical instrument for laparoscopic hysterectomy.

Laparoscopic hysterectomy is a surgical procedure that allows the uterus to be detached from inside the body by laparoscopic instruments while the doctor is viewing the uterus, tubes, and ovaries through a camera attached to a telescope. After the uterus is detached, it is removed through a small incision at the top of the vagina. One advantage of laparoscopic hysterectomy is that the incisions are smaller (½ inch) and much less uncomfortable than that of abdominal hysterectomy. Also, the hospital stay of 1 day and the ability to resume normal activity in about 2 weeks are substantially shorter than for abdominal hysterectomy and slightly shorter than for vaginal hysterectomy.

A laparoscopic hysterectomy is often performed using electrosurgical instruments.

Such an instrument is e.g. known from U.S. Pat. No. 6,176,858 which discloses an instrument and method for separating the uterine body from the cervix during a laparascopic supracervical hysterectomy. However, this device is extremely complex and its use is quite time consuming. A wire segment must be placed around the cervix inside the body. Then one end of the wire segment must be mated to the other (fasteners are provided) before the loop is pulled to remove the cervix from the uterine body. As the loop cannot be cinched, the cutting operation requires that the wire loop must all be pulled forward, thereby potentially contacting other tissue.

Electrosurgical instruments and techniques are widely used in surgical procedures because they generally reduce patient bleeding and trauma associated with cutting operations, as compared with mechanical cutting. Conventional electrosurgical procedures may be classified as operating in monopolar or bipolar mode.

Monopolar techniques rely on external grounding of the patient, where the surgical device defines only a single electrode pole. Bipolar devices have two electrodes for the application of current between their surfaces.

Conventional electrosurgical devices and procedures, however, suffer from a number of disadvantages. For example, these electrosurgical instruments typically operate by creating a voltage difference between the active electrode and the target tissue, causing an electrical arc to form across the physical gap between the electrode and the tissue. At the point of contact of the electric arcs with the tissue, rapid tissue heating occurs due to high current density between the electrode and the tissue. This high current density causes cellular fluids to rapidly vaporize into steam, thereby producing a “cutting effect” along the pathway of localized tissue heating. Thus, the tissue is parted along the pathway of evaporated cellular fluid, inducing undesirable collateral tissue damage in regions surrounding the target tissue.

Further, monopolar electrosurgical devices generally direct electric current along a defined path from the exposed or active electrode through the patient's body to the return electrode, the latter externally attached to a suitable location on the patient. This creates the potential danger that the electric current will flow through undefined paths in the patient's body, thereby increasing the risk of unwanted electrical stimulation to portions of the patient's body.

In addition, since the defined path through the patient's body has relatively high electrical impedance, large voltage differences must typically be applied between the return and active electrodes in order to generate a current suitable for ablation or cutting of the target tissue. This current, however, may inadvertently flow along body paths having less impedance than the defined electrical path, which will substantially increase the current flowing through these paths, possibly causing damage to or destroying surrounding tissue.

Furthermore, insulation failure and capacitive coupling cause electrical current to come in contact with non-target tissue, causing unintended injury. Unlike external skin burns at the site of the patient return electrode, which are usually recognized immediately following a case, stray electrosurgical burns occur outside the view of the laparoscope, unbeknownst to the surgeon. Unaware that electrical currents may be dangerously straying, the surgeon cannot intervene to prevent injury, let alone treat such injury

Bipolar electrosurgical devices have an inherent advantage over monopolar devices because the return current path does not flow through the patient. In bipolar electrosurgical devices, both the active and return electrode are typically exposed so that both electrodes may contact tissue, thereby providing a return current path from the active to the return electrode through the tissue.

One drawback with this configuration, however, is that the return electrode may cause tissue desiccation or destruction at its contact point with the patient's tissue. In addition, the active and return electrodes are typically positioned close together to ensure that the return current flows directly from the active to the return electrode. The close proximity of these electrodes generates the danger that the current will short across the electrodes, possibly impairing the electrical control system and/or damaging or destroying surrounding tissue.

In addition, conventional electrosurgical methods are generally ineffective for ablating certain types of tissue, and in certain types of environments within the body. For example, loose or elastic tissue, such as the uterus, is difficult to remove with conventional electrosurgical instruments because the flexible tissue tends to move away from the instrument when it is brought against this tissue. Since conventional techniques rely mainly on conducting current through the tissue, they are not effective when the instrument cannot be brought adjacent to, or in contact with, the elastic tissue for a sufficient period of time to energize the electrode and conduct current through the tissue.

Thus, it is a first aspect of the present invention to provide an instrument of the kind mentioned in the opening paragraph, which will reduce undesirable tissue damage in regions surrounding the target tissue during use.

It is a second aspect of the present invention to provide an device of the kind mentioned in the opening paragraph, which will reduce the risk of capacitive coupling.

It is a third aspect of the present invention to provide an instrument of the kind mentioned in the opening paragraph, which is simple and reliable in design and in operation.

It is a fourth aspect of the present invention to provide an instrument of the kind mentioned in the opening paragraph, which is adapted for the precise removal or modification of the uterus at a specific location, wherein the uterus can be ablated, severed, resected, contracted, and/or coagulated, with minimal, or no, collateral tissue damage.

It is a fifth aspect of the present invention to provide an instrument of the kind mentioned in the opening paragraph, which is conductive to long-term storage and inexpensive to manufacture so as to enable its disposal after a single use.

This is achieved according to the present invention, as the electrosurgical instrument further comprises a second electrode, which in a first mode is positioned within the shaft, and in a second active mode are expanded from the shaft.

Thereby is not only provided the unique feature, that the second electrode is embedded in the shaft during the placement of the instrument, ensuring that the second electrode will not disrupt any tissue during the insertion of the instrument, but also, that said electrode can be expanded ensuring a large surface area during use, which will eliminate undesirable tissue damage in regions surrounding the target tissue during use. Furthermore, the instrument according to the invention is extremely simple to operate and the instrument therefore provides an improvement over prior art monopolar and bipolar treatment methods, both in decreasing treatment time, and simplifying control of the treatment.

The electrosurgical instrument has an expandable and contractible thin wire loop at its distal end. The loop expands when pushed out of the distal end of the shaft and contracts when withdrawn into distal end. A manual actuator that is attached to the wire loop permits the operating physician to push the loop out of shaft and to withdraw the loop again into the shaft. Said actuator can be arranged in a number of ways as will be evident for at person skilled in the art.

Conventional means will furthermore be provided for electrical actuation of loop by way of a voltage supply. Such means are well known for at person skilled in the art.

The second electrode will in its expanded position, i.e. in the second position, constitute the return electrode. In this position the second electrode will be substantially larger in diameter and surface area than the first electrode, thereby further reducing tissue desiccation or destruction at its contact point with the patient's tissue. In addition, the danger that the current will short across the electrodes is eliminated. It is preferably, that the surface area of the second electrode in relation to the first electrode is at least about from 8:1 to 2:1 and most preferably 4:1, as the inventors surprisingly have found, that this ratio is extremely beneficial for use in electrosurgical instruments.

When the loop is properly positioned, a plunger that is attached to the wire loop permits the operating physician to activate the second electrode, i.e. pushing the second electrode out of shaft, expanding said electrode. Said plunger can be arranged in a number of ways as will be evident for at person skilled in the art. Conventional means will furthermore be provided for electrical actuation of loop by way of a voltage supply. Such means are well known for at person skilled in the art.

Preferably the electrosurgical instrument according to the invention contains a second electrode having multiple retractable/expandable electrodes that are kept inside the shaft until its loop is positioned around the tissue of interest, e.g. the uterus. By having a number of different electrodes, this will ensure, that the return current is more evenly distributed further reducing the risk of damage to the surrounding tissue.

In one embodiment of the present invention the second electrode may be an electrically conductive grasper positioned at the distal end of the shaft and the instrument has means for opening and closing the grasper. The electrically conductive grasper may therefore be used to grasp the biological tissue that is to be cut using the instrument, where the grasper ensures that the biological tissue is in optimal electrical communication with the electrosurgical instrument.

Furthermore, the grasper may be used to grasp and move bodily tissue, which may be surgically removed, within the bodily cavity without any application of electrical current. The grasper may advantageously be used to remove severed tissue from the bodily cavity, via the surgical incision in the skin surface.

In order to ensure that the electrosurgical instrument is capable of entering a bodily cavity during a laparoscopic surgery the first and/or the second electrode may be expandably and retractably positioned inside the shaft where the shaft has means for insulating the first and the second electrodes from each other. The shaft may be divided into two separate passages where one passage houses the first electrode and the other passage houses the second electrode. The first and the second electrodes may be separately expanded or retracted into the shaft independently from each other.

In a preferred embodiment the second electrode comprises a number of section each having a bend, arranged so that each section act as arms which tend to spring outwardly away from the shaft and toward the surrounding tissue, when the actuator move the second electrode from the first position to the second position, where the sections will have the shape of a substantially symmetric arrangement of substantially evenly spaced distal section ends, whereby a uniformly spaced second electrode is provided effectively ensuring that undesirable collateral tissue damage in regions surrounding the target tissue is eliminated.

Insulation is preferably provided along the sections in order to prevent unintended electrical coupling between the sections.

The sections can e.g. be strip-shaped with a rectangular cross section where is bend is in the form of an arc. The rectangular cross section provides increased resistance to bending in the width dimension but allows bending more freely in the thickness dimension. This strip-shaped configuration provides increased stability in the lateral direction while allowing the necessary bending in the radial direction.

Different numbers of sections can be arranged in the shaft of the instrument. The number of sections is in principal only limited by the number that can be extruded in the shaft.

Alternately, the sections are formed straight and connected to a ring placed instead the tube, such that each section tend to expand or spring radially outward from the distal end when the plunger moves the second electrode from the first to the second mode.

In an alternative embodiment the sections are leaf shaped which can be opened-out or unfolded from the shaft, said leaf shaped electrode will in the second active mode assumes the shape of a flower.

The sections of the second electrode does not necessarily needs to be symmetrical arranged around the axle of the shaft. The sections of the second electrode can within the scope of protection just as well be placed asymmetrical. This can in some instances obtain the additional benefit that the return current is catered at one place. Furthermore, the asymmetrical electrode can be arranged in such a way that extra space is provided to surrounding tissue during use of the instrument e.g. an operation, further eliminating the damages to surrounding tissue.

The second electrode can have any kind of shape and design within the scope of protection, as long as the second electrode in one way or another can have a first folded design in the resting position and a second unfolded design when the instrument is used for cutting.

The electrosurgical instrument according the invention further comprises an actuator, preferably positioned at the distal end of the instrument and arranged to permit expansion and contraction of the loop, said loop is preferably arranged for being able to expand sufficiently to fit over the top of the human uterus.

In a preferred embodiment said loop consists of a first electrically conductive material that is surrounded by an insulating member substantially along its entire length, but having an opening in the insulating member at a predetermined location. The opening exposes the electrically conductive inner member at that location. The size of the opening must be sufficient to provide a suitable cutting length, preferably on the order of 0.3-1 cm in length.

The electrically conductive inner member forms the active element of the bipolar pair and is electrically coupled via one or more cable to a source of energy.

The first and second electrode can preferably be made of a metal that is able to resist the high current levels and heat obtained during use of the instrument. Suitable metals include platinum, platinum-iridium, tungsten, and molybdenum we well as numerous alloys of the same.

The insulating member is preferably adapted to provide rigidity to the loop in order to prevent the more distal portion of said expanded loop from being pulled down by gravity.

An operation will normally start with the opening of at least one port into the patient e.g. the patient's abdomen. The distal end of the instrument is passed through the port and into the abdominal cavity and the thin wire loop is expanded and passed over the top of the uterus. In order to perform a supercervical hysterectomy the thin wire loop is positioned at the boundary between the cervix and the body of the uterus and contracted together, to sever the body of the uterus from the cervix. At this point the plunger at the proxmial end of the shaft will be activated and expand the second electrode from the first to the second position, whereby the surface area and diameter of the second electrode will be substantially larger than the first electrode.

As will be evident for the person skilled in the art, when energy is provided to the instrument according to the invention, the first electrode will work as the active electrode and the second electrode will work as the return electrode.

The loop is then electrically actuated and is further cinched inwardly until the uterine body has been severed from the cervix. The second electrode is then moved from the second expanded position to the first retracted position by activation the plunger, and the instrument is then removed from the patient.

It is preferably, that the surface area of the second electrode in relation to the surface area of the first electrode is at least about from 8:1 to 2:1 and most preferably 4:1, as the inventors surprisingly have found, that this ratio is extremely beneficial for use in electrosurgical instruments.

In an even simpler embodiment according to the invention the first and second electrode are both part of the loop, extending at least along the length of the loop.

In order to provide the first and second electrode, the insulation is removed from the first electrode at one position and from the second electrode at a different position.

The first electrode is preferably placed at the distal end of the loop, in order to ensure an optimal cutting ability of the instrument, as most force during the contraction of the loop will be placed at the distal end of the loop. However other designs can have other benefits and the two electrodes can e.g. be connected at the tip of the loop.

In order to simplify the placement of the loop around a specific biological tissue, the loop may comprise at least two elongated electrically conductive blades, each having a proximal and a distal end, where the distal ends are in electrical communication via an electrically conductive wire constituting the first electrode. The elongated blades preferably have a insulating cover, such that the risk of application of electrical current via the blades is minimized. The elongated blades provide stiffness to the loop, such that the loop may be easily manoeuvred inside the bodily cavity, as the elongated blades do not bend or buckle when coming in contact with an object within the cavity.

In a preferred embodiment of the present invention, the electrically conductive wire may be surrounded by an insulating member substantially along its entire length, but having an opening in the insulating member at the tip. This means that the electrical current flowing into the biological tissue, may be concentrated in a small surface area which increases the surgical precision of the wire.

In a further development of this embodiment the first electrode is placed adjacent to the second electrode, and both electrodes are twisted in the longitudinal direction, and said twisted electrode constitutes the loop. When the insulation is removed from a part of the loop the two electrodes will be exposed, alternate in the longitudinal direction, i.e. the two electrodes will be exposed one after the other throughout the loop.

In a still further embodiment according to the invention the loop is prepared by at least two first electrodes and at least two second electrodes, placed in a quadrant, where the corresponding electrode are placed diagonal in respect to each other, divided by an insulation. Again the entire construction is twisted constituting the loop. When part of the insulation is removed two electrodes will be exposed, alternate in the longitudinal direction, i.e. the two electrodes will be exposed one after the other throughout the loop. This embodiment according to the invention has the advantage that the active and return electrode will be exposed almost adjacent to each other elimination the danger that the electric current will flow through undefined paths in the patient's body, and the risk of unwanted electrical stimulation to portions of the patient's body.

The foregoing and other objectives, features and advantages of the invention will be more readily understood upon consideration of the following detailed description of the invention, taken in conjunction with the accompanying drawings.

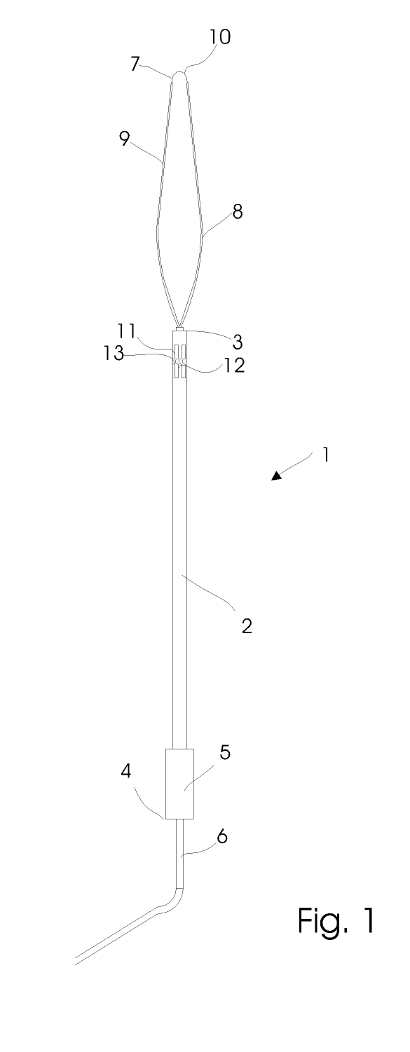

FIG. 1 is a representation of a first embodiment of an electrosurgical instrument according to the invention seen in a first position,

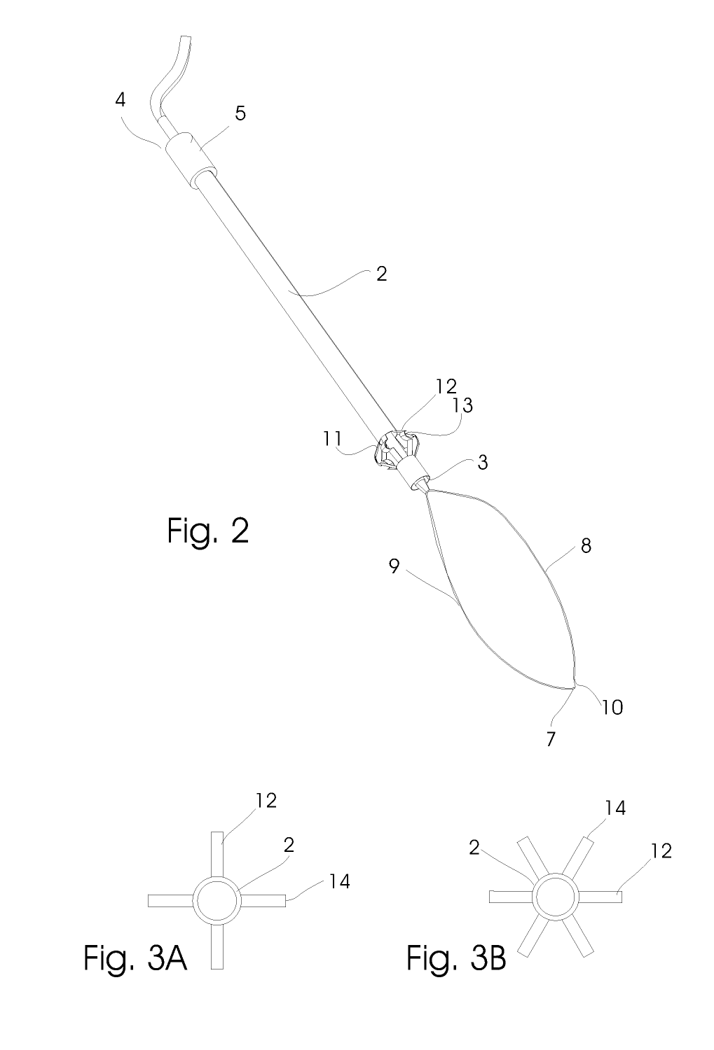

FIG. 2 shows a perspective view of the same embodiment in a second position,

FIG. 3 shows a cross-section along the line III-III in FIG. 2,

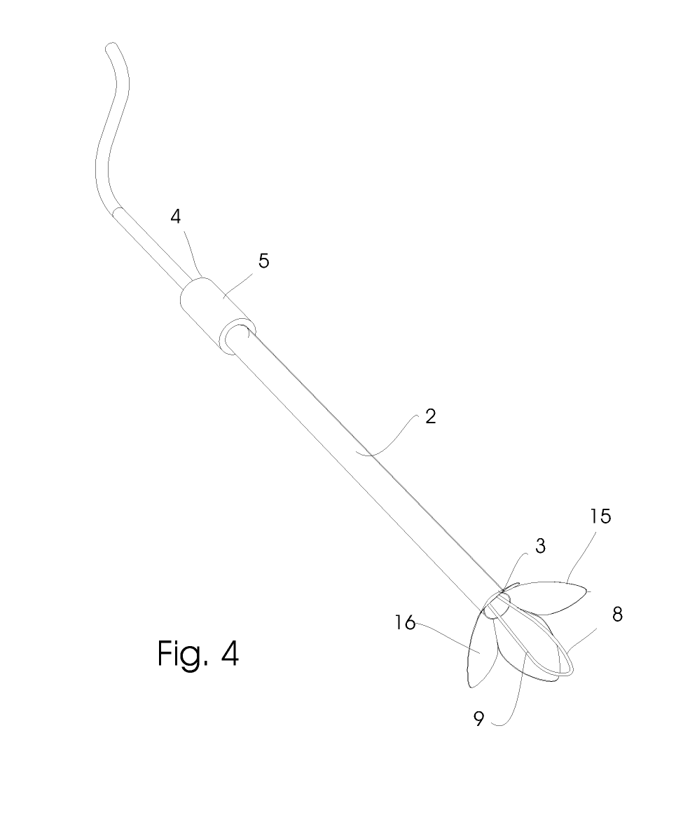

FIG. 4 shows a perspective view of a second embodiment of a electrosurgical instrument according to the present invention,

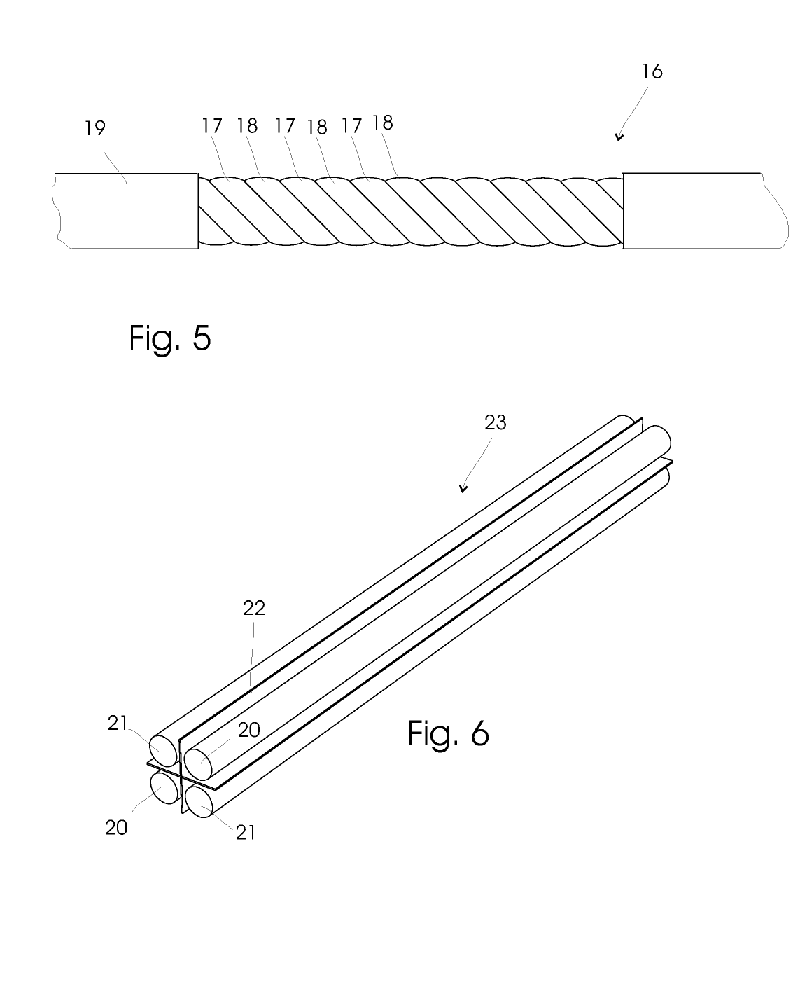

FIG. 5 shows one embodiment of a loop to the electrosurgical instrument according to the present invention,

FIG. 6 shows a second embodiment of a loop to the electrosurgical instrument according to the present invention,

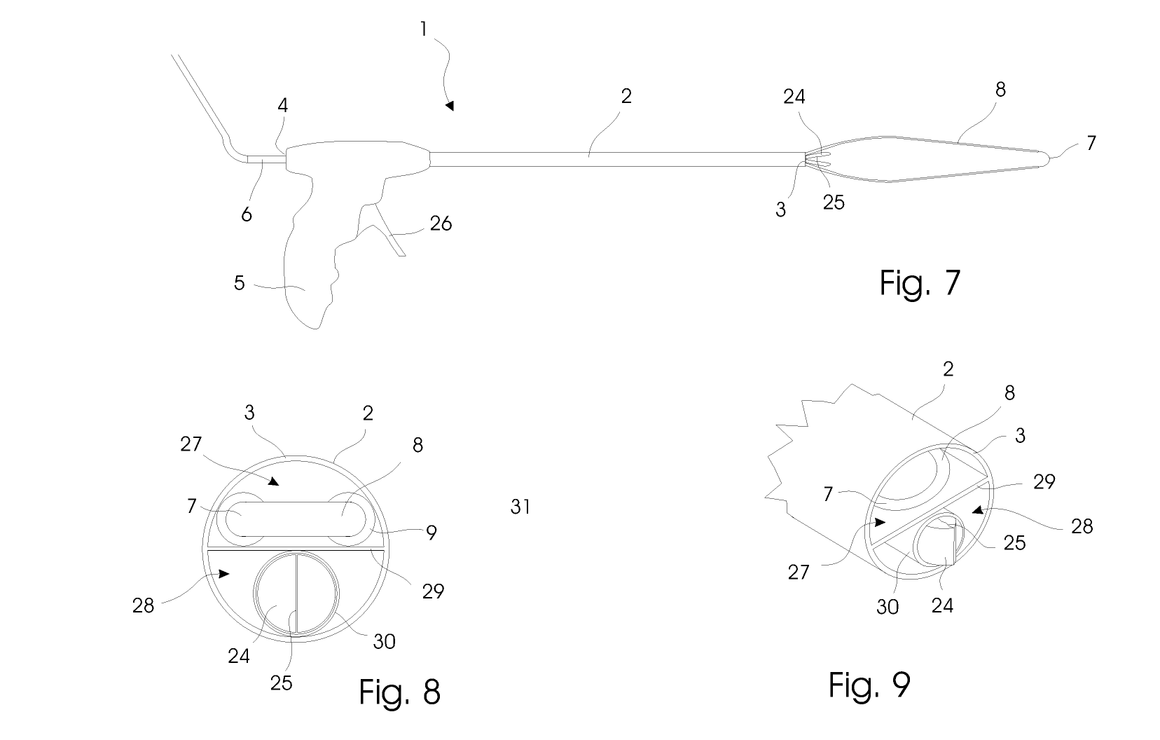

FIG. 7 shows a side view of a third embodiment of a electrosurgical instrument according to the present invention, where the second electrode is a grasper,

FIG. 8 shows a front view of the distal end of the electrosurgical instrument, according to a third embodiment having a loop and a grasper in a retracted position,

FIG. 9 shows a perspective view of the distal end of the electrosurgical instrument in a third embodiment of the instrument, having a loop and a grasper in a retracted position,

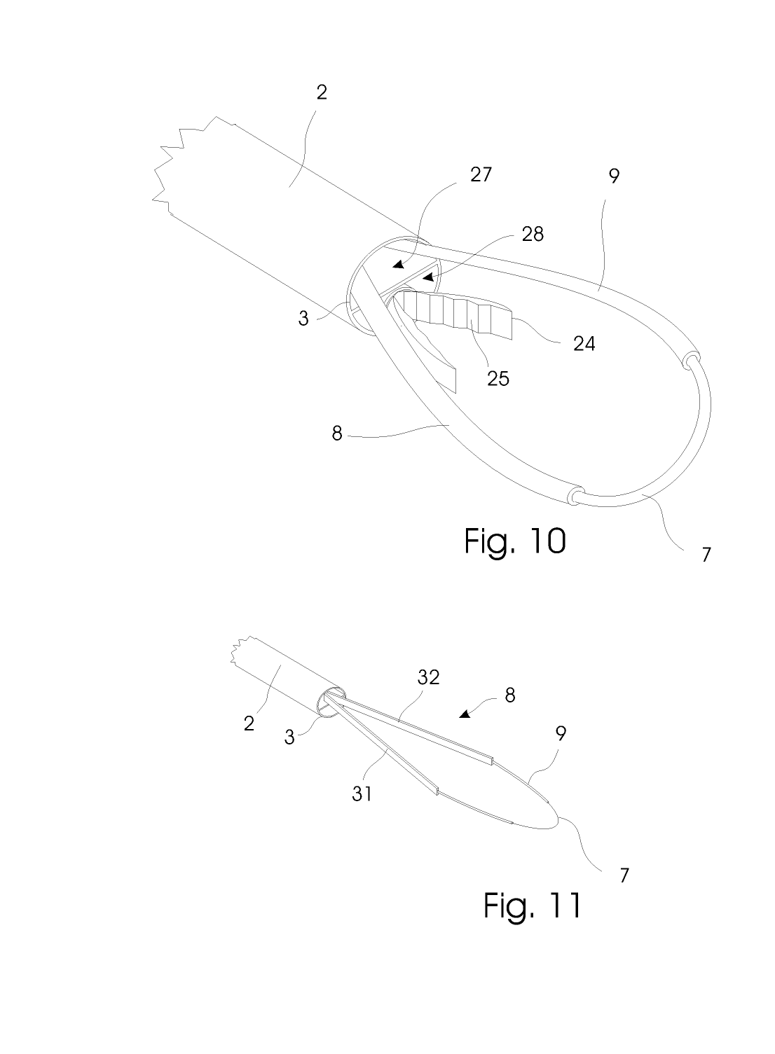

FIG. 10 shows a perspective view of the distal end of the electrosurgical instrument in a third embodiment having a loop and a grasper in a extended position, and

FIG. 11 shows a third embodiment of a loop to the electrosurgical instrument according to the present invention.

FIG. 1 shows a first embodiment 1 of an electrosurgical instrument according to the present invention. In the following it is assumed, that the instrument is used for a laparoscopic hysterectomy, however the instrument could evenly well be used for a number of other electrosurgical procedures, such as removal of the cervix or ovaries.

The electrosurgical instrument 1 comprises a hollow shaft 2 having a distal 3 and a proximal end 4. The proximal end 4 expands into a manual handle 5, adapted to be held and manipulated by a physician.

A bar 6 is slideable positioned within the shaft 2. Said bar comprises an electrically conductive wire 7, forming a first electrode, which at the distal end of the bar 6 expands into a loop 8. The loop is surrounded by an insulating member 9 substantially along its entire length, but having an opening in the insulating member at the tip 10. When the bar 6 is displaced axially in the hollow shaft 2 the wire loop 8 will expand when pushed out of distal end 3 and contract when withdrawn into distal end 4.

At the proximal end the wire 7, is connected to a standard electrical jack (not shown) adapted to connect with a voltage source.

Near the distal end 3 of the shaft 2, a second electrode 11 is positioned, constituting the return electrode. Said electrode comprises a number of retractable/expandable electrodes 12 that are kept inside the shaft 2 until the loop 8 is positioned around the tissue of interest, e.g. the uterus. By having a number of different electrodes, this will ensure, that the return current is more evenly distributed further reducing the risk of damage to the surrounding tissue.

Each electrode 12 is strip-shaped with a rectangular cross section having a bend 13. The rectangular cross section provides increased resistance to bending in the width dimension but allows bending more freely in the thickness dimension. This strip-shaped configuration provides increased stability in the lateral direction while allowing the necessary bending in the radial direction.

Thereby will each electrode 12 act as arms which tend to spring outwardly away from the shaft 2, when the handle 5 is manipulated into forcing the second electrode 11 from the first position to the second position.

As best seen in FIG. 3, each section 12 will then obtain the shape of a substantially symmetric arrangement of substantially evenly spaced distal section ends 14, whereby a uniformly spaced second electrode 11 is provided effectively ensuring that undesirable collateral tissue damage in regions surrounding the target tissue is eliminated.

The second electrode will in the expanded position shown in FIG. 2, have a substantially larger diameter and surface area than the first electrode 6, thereby reducing tissue desiccation or destruction at its contact point with the patient's tissue. Different numbers of sections 12 can be arranged in the shaft 2 of the instrument 1. The number of sections 12 is in principal only limited by the number that can be extruded in the shaft. The embodiment shown in FIG. 3A has four sections 12, whereas the embodiment in FIG. 3B has six.

Insulation (not shown) is provided along the sections in order to prevent unintended electrical coupling between the sections.

During the operation the physician will open at least one port into the patient e.g. the patient's abdomen. The distal end 3 of the instrument 1 is passed through the port and into the abdominal cavity and the thin wire loop 6 is expanded and passed over the top of the uterus.

In order to perform a supercervical hysterectomy the thin wire loop is positioned at the boundary between the cervix and the body of the uterus and contracted together, to sever the body of the uterus from the cervix. At this point the actuator at the proxmial end 4 of the shaft 2 will be activated and expand the second electrode 11 from the first to the second position, whereby the surface area and diameter of the second electrode 11 will be substantially larger than the first electrode 6.

When current is added to the instrument 1 the first electrode 6 will work as the active electrode and the second electrode 11 will work as the return electrode.

The loop 8 is then electrically actuated and is further cinched inwardly until the uterine body has been severed from the cervix. The second electrode 11 is then moved from the second expanded position to the first retracted position by activation the plunger, and the instrument 11 is then removed from the patient.

FIG. 4 shows a second embodiment of the instrument 1 according to the invention. For the most part, the instrument shown in FIG. 4 corresponds to the instrument in FIG. 1-3, and identical parts have been given the same reference number. In this embodiment the second electrode 15 has three leaf shaped sections 16 which can be opened-out or unfolded from the shaft 2. As can be seen said leaf shaped electrode 15 will in the second active mode assumes the shape of a flower.

FIG. 5 shows a section of one embodiment of a loop 16 according to the invention. In this embodiment the first electrode 17 and the second electrode 18 are twisted along the entire length of the loop 16. When the insulation 19 is removed from e.g. the tip of the loop the two electrodes 16, and 17 will be exposed repeatedly one after the other, revealing both a This loop will provide a very simple and inexpensive design of the loop.

FIG. 6 shows a section of a second embodiment of the loop in FIG. 5. For clarity reasons the different electrodes is not shown twisted as in FIG. 5. The loop is prepared by placing two first electrodes 20 and two second electrodes 21 in a quadrant, where the corresponding electrode are placed diagonal in respect to each other. The electrodes are divided by an insulation 22, said insulation will also cover the entire twisted contraction (not shown)

When the entire construction is twisted (not shown) and a part of the insulation removed the electrodes will be exposed, alternate in the longitudinal direction, i.e. the two electrodes will be exposed one after the other throughout the loop, effectively ensuring that undesirable collateral tissue damage in regions surrounding the target tissue is eliminated.

The two embodiments of the loop shown in FIGS. 5 and 6 have the further advantage, that the instrument according to the invention is extremely simple to both operate and manufacture. Therefore, the instrument according to the invention provides an improvement over prior art monopolar and bipolar treatment methods, both in decreasing treatment time, and simplifying control of the treatment.

FIG. 7 shows a side view of a third embodiment of a electrosurgical instrument 1, where the second electrode is a grasper. The electrosurgical instrument comprises a hollow shaft 2, which houses the electrosurgical loop 8 and a grasper 24, which are extendable and retractable into the hollow shaft, at the distal end. The loop 8 may be extended and retracted into the shaft 2 using the slideable bar 6 positioned inside the shaft 2, at the proximal end 4.

The electrosurgical instrument, may be single handed held using the manual handle 5, which is shaped and formed substantially as a pistol grip, having a trigger mechanism 26, which may used to extend and retract the grasper 24 out of the distal end 3 of the shaft 2. The grasper 24, may advantageously be provided with a serrated gripping surface 25 for increasing the friction between a gripped material (not shown) and the grasper.

FIG. 8 and FIG. 9 shows a front and frontal perspective view, respectively, of the distal end 3 of the electrosurgical instrument, according to a third embodiment having a loop 8 and a grasper 24 in a retracted position. The shaft 2, is divided into a first 27 and a second pathway 28, which are isolated from each other along the longitudinal axis of the shaft 2 using a divider 29. The loop 8 and the grasper 24 may be extended and retracted individually from the distal end 3 their respective pathway 27,28. In a retracted position the gripping surface 25 of the grasper 24 is forced together via a tubular housing 30, which has an inner diameter, which is substantial to the outer diameter of the grasper 24 in a closed position.

FIG. 10 shows a perspective view of the distal end 3 of the electrosurgical instrument in a third embodiment having a loop 8 and a grasper 24 in an extended position. The loop 8 is extended from its pathway 27, in a position where the biological tissue to be cut may be snared into the loop 8. The grasper 24 is extended out of its pathway 28 and its tubular housing 30, where the gripping surface 25 is in a spread out position and is ready for grabbing the biological tissue, being snared by the loop 8.

FIG. 11 shows a third embodiment of a loop 8 to the electrosurgical instrument according to the present invention. The loop 8 comprises two stiff blades 31,32 which may be extended and retracted from the distal end 3 of the shaft 2, an electrically conductive wire 7 and insulating member 9 surrounding a part of the wire 7. The blades 31,32, are advantageously insulated by a coating (not shown) such that the electrosurgical current may not flow directly from the blades 31,32, and only via the wire 7 which is in electrical communication with the blades. Advantageously, the electrical flow only flows via areas of the wire, which are not insulated by the insulating member 9. The stiff blades 31,32 in an extended position make the maneuvering of the loop 8 easier, as the loop does not buckle or bend.

The terms and expressions which have been employed in the foregoing specification are used as terms of description and not of limitation, and there is no intention, in the use of such terms and expressions, of excluding equivalents of the features shown and described or portions thereof, it being recognized that the scope of the invention is defined and limited only by the claims which follow.

1.-14. (canceled)

15. An electrosurgical instrument comprising a shaft sized to fit through a laparascopic port and having proximal and distal ends; an expandable and contractible loop positioned to controllably emerge from the distal end of the shaft and defining a first electrode; at least one second electrode which in a first position is positioned within the shaft and in a second position is expanded from the shaft; and means for expanding or retracting the second electrode.

16. The electrosurgical instrument according to claim 15, wherein the means for expanding or retracting the second electrode is a bar that is slideably positioned in the shaft.

17. The electrosurgical instrument according to claim 15, wherein the second electrode is a return electrode which in the second position is substantially larger in diameter and surface area than the first electrode.

18. The electrosurgical instrument according to claim 15, wherein the second electrode is a electrically conductive grasper positioned at the distal end of the shaft and the instrument includes means for opening and closing the grasper.

19. The electrosurgical instrument according to claim 15, wherein the first or second electrode or both is expandably and retractably positioned inside the shaft and wherein the shaft has means for insulating the first and second electrodes from each other.

20. The electrosurgical instrument according to claim 15, wherein the second electrode comprises a number of sections, with each section comprising a preformed bend arranged for enabling each section to expand outwardly when the second electrode is moved from the first position to the second position, whereby each section end moves away from each other to form a substantially symmetric arrangement of substantially evenly spaced distal section ends.

21. The electrosurgical instrument according to claim 20, wherein each bend is in the form of an arc.

22. The electrosurgical instrument according to claim 15, wherein the second electrode has an essentially a flat structure which can be opened-out or unfolded from the shaft, which in the first mode assumes essentially the same shape as the longitudinal axis of the shaft and in the second mode assumes the shape of a flower.

23. The electrosurgical instrument according to claim 15, further comprising a handle positioned at the distal end of the instrument and arranged to permit expansion and contraction of the loop.

24. The electrosurgical instrument according to claim 15, wherein the loop is arranged to expand sufficiently to fit over the top of the human uterus.

25. The electrosurgical instrument according to claim 15, wherein the loop comprises insulation of a flexible dielectric material.

26. The electrosurgical instrument according to claim 15, wherein the loop comprises at least two elongated electrically conductive blades each having proximal and distal ends, where the distal ends are in electrical communication via an electrically conductive wire constituting the first electrode.

27. The electrosurgical instrument according to claim 26, wherein the electrically conductive wire is surrounded by an insulating member substantially along its entire length, but having an opening in the insulating member at the tip.

28. The electrosurgical instrument according to claim 15, wherein the loop comprises at least first and second electrodes twisted around each other in the longitudinal direction.
